(19) United States
(12) Patent Application Publication
(10) Pub. No.: US 2006/0175425 A1
(43) Pub. Date: Aug. 10, 2006

(54) Dispensing device

(75) Inventors: Thomas McGee, Nyack, NY (US); Guy Edward Naish, Oxfordshire (GB); Colin Brown, Berkshire (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 10/512,666

(22) PCT Filed: Apr. 25, 2003
(86) PCT No.: PCT/CH03/00270

§ 371 Date: Dec. 8, 2004

Related U.S. Application Data
(60) Provisional application No. 60/377,711, filed on May 3, 2002.

(51) Int. Cl. A61L 9/04

(52) U.S. Cl. 239/44

(57) ABSTRACT

A device for dispensing a volatile liquid as a vapor to an ambient environment, i.e., a room or vehicle interior, which device comprises a reservoir of the volatile liquid, and a porous element which permits the passage of vapor therethrough but does not permit the passage of the volatile liquid therethrough, characterized in that the device greatly decreases the likelihood of undesired spillage of the volatile liquid from the device when the device held in any orientation.

The present invention relates to improved dispensing devices. More particularly the present invention relates to dispensing devices useful for the delivery of a volatile or evaporable material to an ambient environment, such as a room, or interior of a vehicle which dispensing devices exhibit a reduced tendency for the undesired spillage of the volatile liquid from the device when the device held in any orientation.

Various devices useful for the delivery of volatile materials such as fragrances, odor masking agents, insecticides, medicaments as well as other volatile materials which may have a cosmetic, insecticidal or medical effect are known in the art. One particular class of such devices are those which are used for the delivery of any liquid composition which is volatile, or evaporable, to an ambient environment. Typically, such includes a reservoir or other container capable of containing an amount of such a volatile material, which reservoir or other container includes a neck through which protrudes a wick. The wick operates to transport the liquid by means of capillary action from the interior of the reservoir to the ambient environment, into which it evaporates or volatilizes. Such devices are simple, but frequently effective. In certain embodiments, such devices are supplied as part of a larger apparatus, which may provide an external feature for static diffusion systems or it may include a means of diffusing the volatile liquid, such as a heat source, which may surround some or part of the wick, and when heated induces more rapid volatilization of the liquid, or a fan that causes more rapid volatilization of the liquid into the ambient environment.

Nonetheless, such devices are not free from shortcomings. A particular problem which plagues many prior art devices lies in the fact that unwanted leakage of the liquid frequently occurs. Very often, the wick fails to provide a reliable liquid tight seal at the neck of the reservoir and when the reservoir is tilted or inverted, flow of the liquid past the wick and out of the reservoir is known to occur. A further problem is that subsequent to the manufacture of such devices, they may be subjected to rather broad ranges of temperature and pressure subsequent to their manufacture and prior to their initial use by a consumer. Such varying temperatures and pressures are frequently encountered during the shipping and warehousing of such devices to the ultimate retail outlets in which they are sold. These varying pressures and temperatures are often detrimental as elevated temperatures and significant pressure changes may undesirably induce a volatile liquid to evaporate prior to its use by a consumer, recondense in the space between the wick and an overcap which is typically present and form droplets or a pool of free liquid outside of the wick. This is a deleterious effect from a consumer standpoint as upon initial use of such a device the consumer may contact the volatile liquid, and possibly soil their hands and/or stain garments or surfaces due to the presence of this free liquid. A further problem also lies in the fact than in order for such devices to operate effectively, the pressure between the ambient and the interior atmosphere within the reservoir must be equalized, else the capillary action of the wick will prematurely fail. Such premature failure is highly undesirable as the device will fail to operate, even though liquid remains within the interior of the reservoir. Such is highly undesirable both from a technical, and from a consumer standpoint. To solve such a problem, there is frequently the provision of one or more vent holes whereby the pressure can be equalized between both the ambient environment and the interior of the reservoir. However, the provision of one or more such vent holes provides a new problem, in fact such vent holes provide a fluid channel through which the liquid within the reservoir can spill or leak out particularly when the reservoir is inverted. The inversion of the reservoir, is not an unusual condition, and frequently occurs during the transport of a product containing such a reservoir and wick assembly, or during the use of such a device such as by a consumer.

One exemplary device known to the art is described in European patent application EP 1088562. Therein is described a device for the controllable transfer of liquid which device includes a fluid reservoir, a volatile liquid contained within the fluid reservoir, a wick which transports the volatile liquid from the interior of the fluid reservoir to the ambient environment by means of capillary action, said wick being encased within a non-porous holder sleeve. Fluid contact between the wick and the holder sleeve and can be interrupted by tilting or inverting the reservoir, thereby stopping the supply of the volatile liquid to the wick and to the ambient environment. However, this device also has been observed to suffer from “pooling” in that, during transport for after subjecting the device to changes in pressure a quantity although volatile liquid frequently collects upon the upper surface of the holder sleeve. As has been noted previously, this is highly undesirable from a consumer standpoint.

Accordingly, there is a real and urgent need in the art for improved devices useful for the delivery of volatile materials such as fragrances, odor masking agents, insecticides, medicaments as well as other volatile materials which improved devices provide a decreased likelihood of undesired spillage.

The invention therefore provides a dispensing device which comprises a reservoir containing a volatile liquid, and a porous wick characterized in that the device greatly decreases the likelihood of pooling, and/or of undesired spillage of the volatile liquid from the device when the device held in any orientation.

In a first aspect, the dispensing device according to the invention comprises a reservoir containing a volatile liquid, a porous wick having a lower portion adapted to be in fluid communication with said volatile liquid and an upper portion adapted to be in fluid communication with an ambient environment, a lagging in contact with, but preferably surrounding at least a part of the porous wick, a sleeve encasing at least a part of the said lagging, and a disruptable seal which in a first, sealing relationship provides a liquid tight barrier between the wick and the volatile liquid, and in a second, non-sealing relationship permits for fluid communication between the wick and the volatile liquid in the reservoir.

In a second aspect, the dispensing device according to the invention comprises a reservoir containing a volatile liquid, a porous wick having a lower portion in fluid communication with said volatile liquid and an upper portion in communication with an ambient environment, a fitment which includes a vent between the reservoir and the ambient environment, and a lagging interposed between the vent and the ambient environment.

In a third aspect of the invention there is provided a dispensing device which comprises a reservoir containing a volatile liquid, a porous wick having a lower portion adapted to be in fluid communication with said volatile liquid and an upper portion adapted to be in fluid communication with an ambient environment, a lagging in contact with, but preferably surrounding at least a part of the porous wick, a sleeve encasing at least a part of the said lagging said sleeve including a vent between the reservoir and the ambient environment, and a disruptable seal which in a first, sealing relationship provides a liquid tight barrier between the wick and the volatile liquid, and in a second, non-sealing relationship permits for fluid communication between the wick and the volatile liquid in the reservoir.

According to a fourth aspect of the invention there is provided a dispensing device which comprises a reservoir containing a volatile liquid, a porous wick having a lower portion adapted to be in fluid communication with said volatile liquid and an upper portion adapted to be in fluid communication with an ambient environment, a vent between the reservoir and the ambient environment, and an overcap which in a closed relationship provides a vapor tight barrier between the upper portion of the porous wick and the ambient environment as well as provides a liquid tight seal between at least a portion of the overcap and the vent.

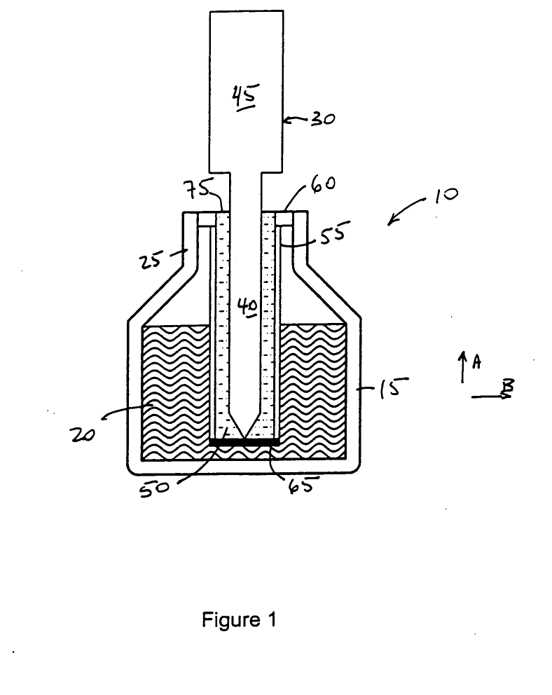

FIG. 1 depicts a dispensing device according to the first aspect of the invention.

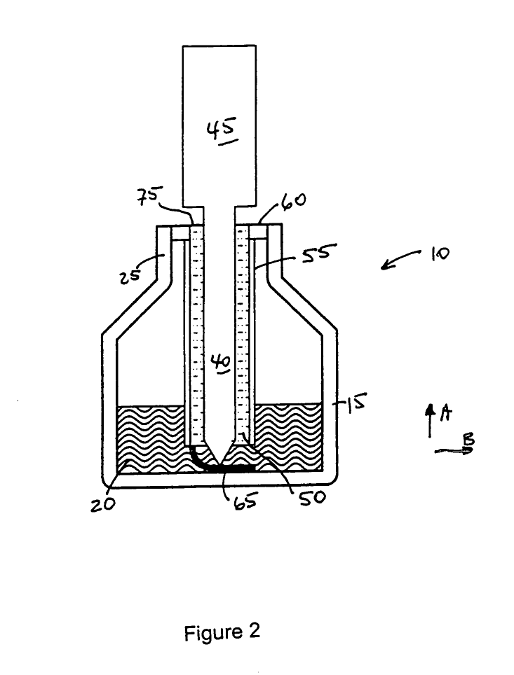

FIG. 2 depicts a further view of the dispensing device according to FIG. 1

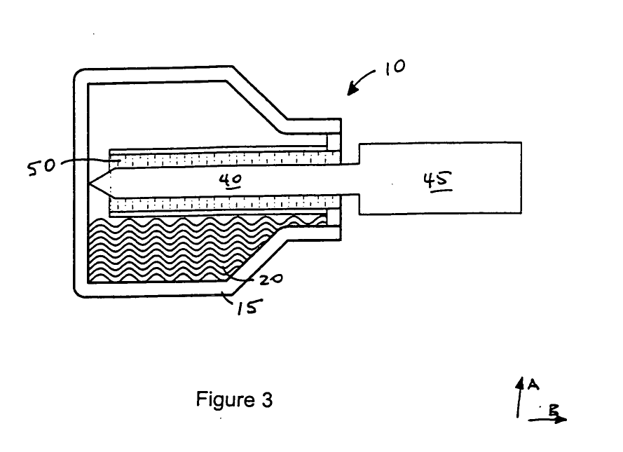

FIG. 3 shows a further view of the dispensing device of FIGS. 1 and 2, but in a horizontal orientation.

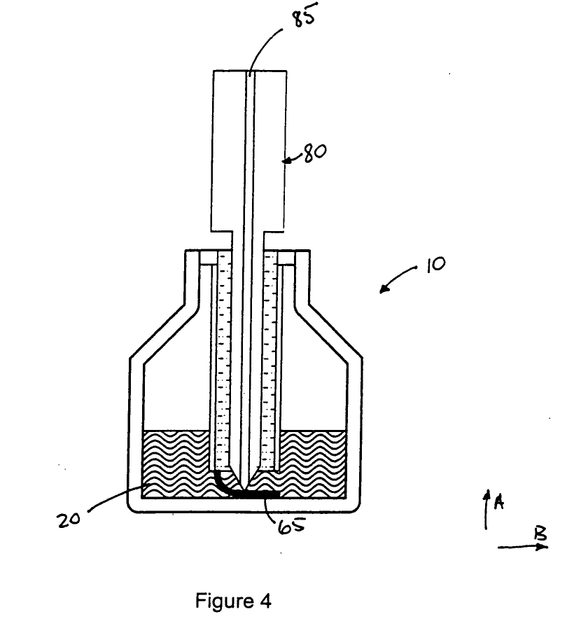

FIG. 4 illustrates an alternative embodiment of FIG. 1 having a different wick configuration.

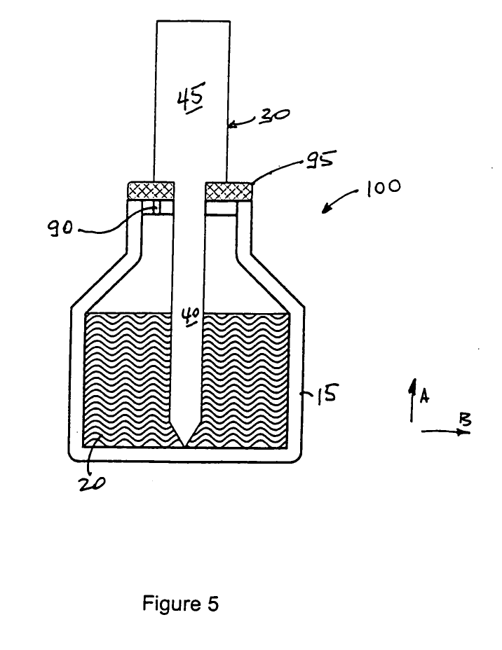

FIG. 5 depicts a dispensing device according to a second aspect of the invention.

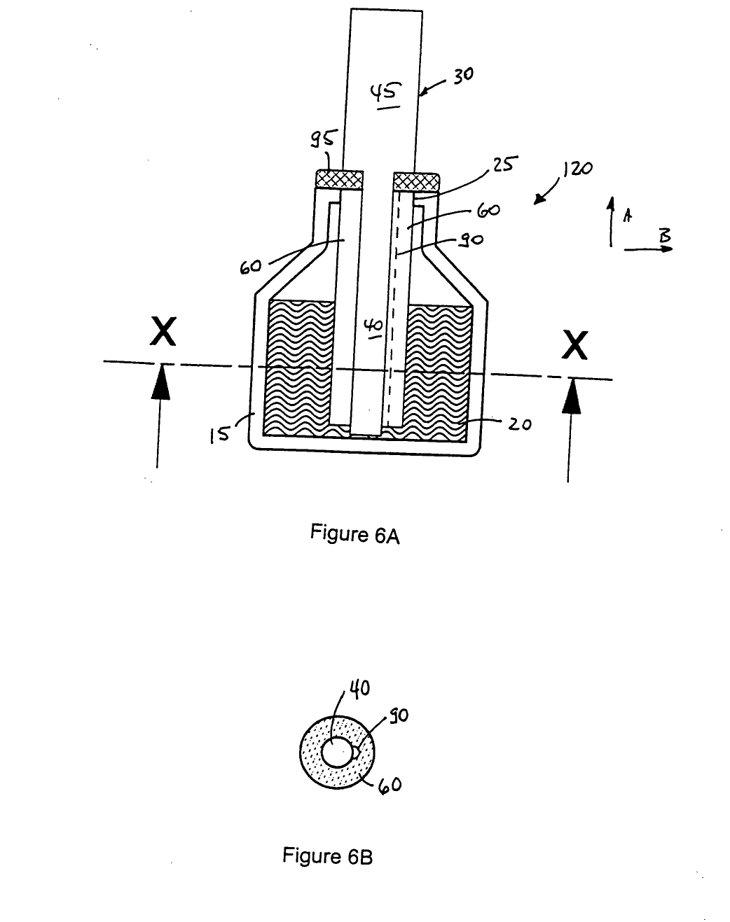

FIG. 6A depicts an alternative dispensing device according to the second aspect of the invention.

FIG. 6B depicts a portion of the dispensing device of the FIG. 6A.

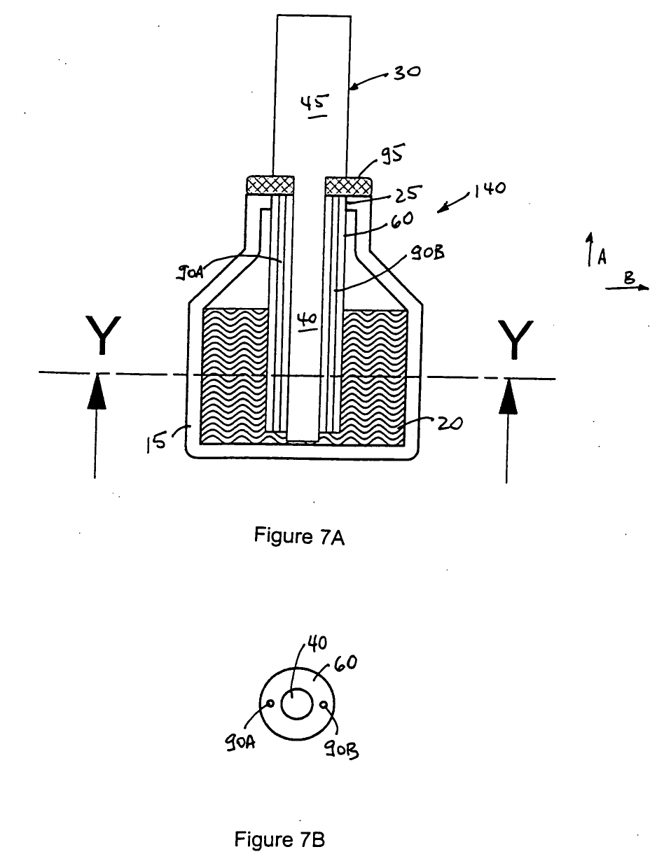

FIG. 7A depicts a further alternative dispensing device according to the second aspect of the invention.

FIG. 7B depicts a portion of the dispensing device of FIG. 7A.

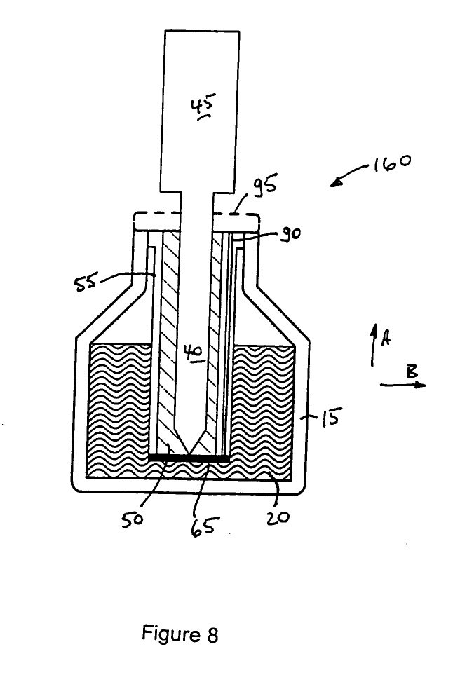

FIG. 8 depicts a dispensing device according to the third aspect of the invention.

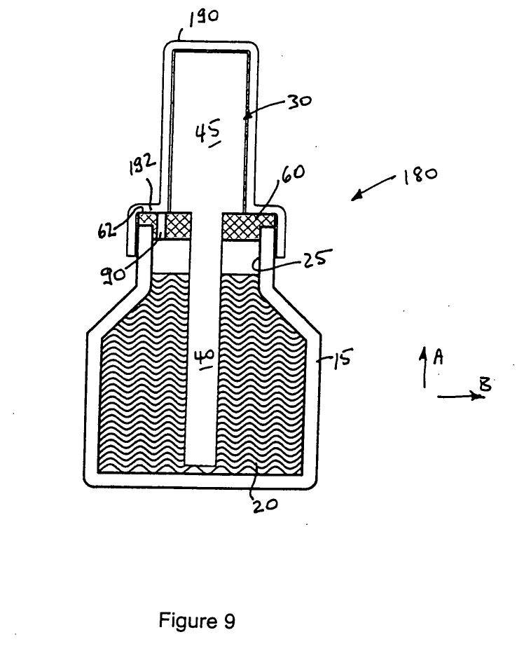

FIG. 9 depicts a dispensing device according to the fourth aspect of the invention.

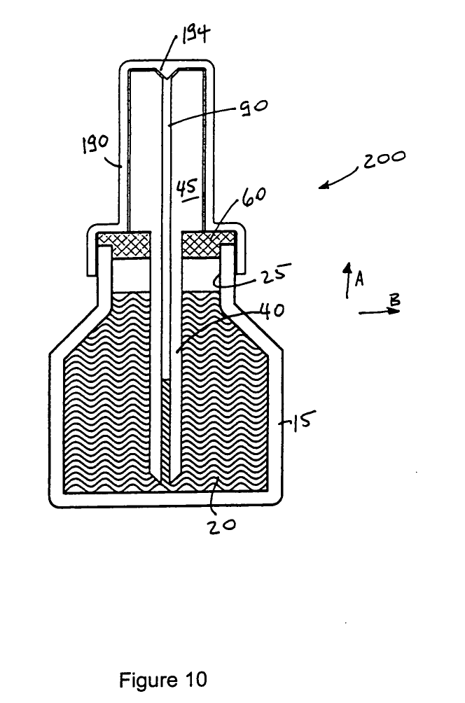

FIG. 10 depicts an alternative embodiment of a dispensing device according to the fourth aspect of the invention.

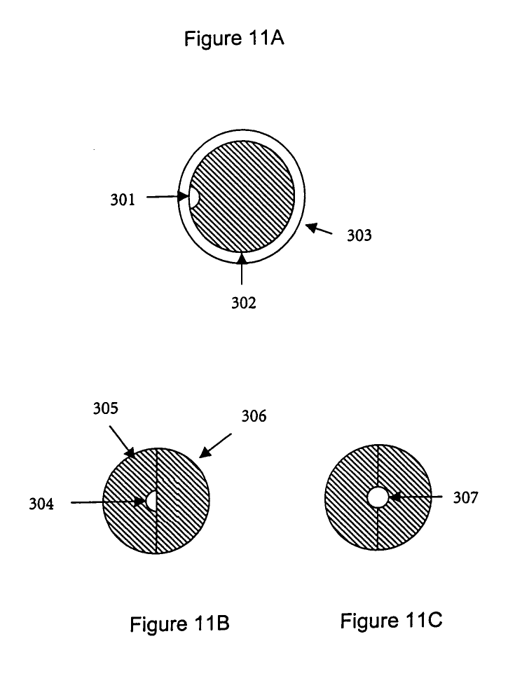

FIG. 11A-C depict a particular design of wick for use in the invention.

Turning now to FIG. 1 therein is depicted a dispensing device 10 according to a first aspect of the invention. The dispensing device 10 includes a reservoir 15 suited for containing quantity of a volatile liquid 20. The reservoir 15 includes a neck 25, through which passes a porous wick 30. The porous wick 30 includes both a lower portion 40 within the interior of the reservoir 15 adapted to be in fluid communication with said volatile liquid, as well as an upper portion 45 exterior of the reservoir 15 which is adapted to be in fluid communication with an ambient environment. The porous wick 30 may be displaced and is moveable within a lagging 50 which surrounds at least a part of the lower portion 40 of the porous wick. The lagging 50 itself is encased by a sleeve 55 which located in the interior of the reservoir 15, and which sleeve 55 is downwardly dependent from a fitment 60 fitted into the neck 25 of the reservoir 15. This sleeve 55 provides a liquid tight barrier between the contents of the reservoir 15, viz., the volatile liquid 20 and the lagging 50 and the wick 30 when a disruptable seal 65 is in a first, sealing relationship. However, when the wick 30 is displaced, disrupting the seal 65 then a second, non-sealing relationship is established which permits for fluid communication between the wick, lagging and the volatile liquid in the reservoir. Desirably, as shown, the lower portion 40 of the porous wick 30 is tapered so to provide facilitate in disrupting, e.g., puncturing of the disruptable seal 65. This second non-sealing relationship is depicted in FIG. 2, and illustrates fluid contact between the volatile liquid 20, and both the lower portion 40 of the porous wick 30, and with the lower end 70 of the lagging 50.

The operative principles of the first aspect of the invention will now be described in more detail with reference to FIGS. 1, 2 and 3.

FIG. 1 illustrates a first preferred embodiment of the invention as discussed previously, which is representative of a dispensing device in its inactivated state. What is meant to be understood by its “inactivated state” is simply that the arrangement of the elements discussed previously are disposed or positioned such that a liquid tight barrier exists between the volatile liquid 20 disposed within the reservoir and between the porous wick and the lagging. In this inactivated state, the dispensing device is eminently suitable for handling, transport, and delivery to the ultimate consumer as there is little or no likelihood that the volatile liquid 20 can leak via the wick or via the lagging. This is assured due to the presence of the disruptable seal 65 which also provides a liquid tight barrier between the volatile liquid 20 disposed within the reservoir and between the porous wick 30 and the lagging 50. It is however to be stated that while the presence of the disruptable seal 65 is preferred, it may be omitted. As can be further seen from FIG. 1, the upper end 75 of the lagging 50 is in fluid communication with the ambient environment; the lagging 50 is sufficiently porous such that the relative pressure between the ambient and the interior atmosphere within the reservoir 15 may be equalized. Such pressure equalization ensures that the capillary action of the wick 30 will not prematurely fail.

FIG. 2 illustrates the first preferred embodiment of the invention in its activated state. What is meant to be understood by its “activated state” is simply that the arrangement of the elements discussed previously are disposed or positioned such that fluid communication exists between the volatile liquid 20 disposed within the reservoir and between the porous wick 30 and the lagging 50. According to this embodiment, disruption of the seal 65 is simply achieved by the displacement of the wick 30 such that the lower portion 40 of the porous wick 30 breaks the seal between the disruptable seal 65 between the volatile liquid 20 disposed within the reservoir and between the porous wick 30 and the lagging 50, and thereby permitting for liquid communication between the volatile liquid 20 and the porous wick 30 and with the lagging 50.

FIG. 3 illustrates a characterizing feature of the first preferred embodiment of the invention. FIG. 3 illustrates the embodiment of FIGS. 1 and 2, in a displaced position such that the dispensing device is in a horizontal orientation as depicted by arrow “B”, as opposed to the prior vertical orientation as depicted by arrows “A” in FIGS. 1 and 2. Arrows “A” and “B” are perpendicular with respect to each other. FIG. 3 is intended to show that, when the disruptable seal 65 is disrupted, the porosity of the wick 30 and the lagging 50 is sufficient such that a sufficient volume of the volatile liquid 20 originally disposed in the reservoir 20 is absorbed into the wick 30 and lagging such that the remaining volume of volatile liquid 20 is insufficient to contact the lagging 50 or the wick 30 when the dispensing device is placed in a horizontal orientation. As such, insufficient volatile liquid 20 is present in the reservoir 15 but not in the wick 30 or lagging 50 such that the volatile liquid 20 may spill or leak when the reservoir is tilted to a horizontal orientation such as shown in FIG. 3, or to an inverted orientation (not shown.)

A further advantage of the arrangement shown in FIGS. 1, 2 and 3 is that as the lagging 50 is in physical contact with the wick 30, when any volatile liquid 20 is absorbed by the lagging 50, physical transfer of the volatile liquid 20 from the lagging 50 to the wick 20 occurs, and the capillary action of the wick 30 ensures that the volatile liquid is transferred to the upper part 45 of the wick 30 from which it may volatilize or evaporate into an ambient environment.

FIG. 4 depicts an alternative embodiment of FIG. 1 having a different wick configuration. Unlike the wick 30 of FIGS. 1, 2 and 3, the wick 80 of FIG. 4 includes a vent channel 85, which provides for airflow between the ambient environment, and the interior of the reservoir 15 such that the relative pressure between the ambient and the interior atmosphere within the reservoir may be equalized. In this embodiment, the vent channel 85 is a hollow channel within the interior of the wick 80 and is located along the length of the wick 80, preferably approximately along the central axis of the wick as shown in FIG. 4.

FIG. 5 illustrates a dispensing device 100 according to a second aspect of the invention. The dispensing device 100 includes many of the same elements illustrated in FIGS. 1-4, including a reservoir 15 containing a volatile liquid 20, a porous wick 30 having a lower portion 40 and an upper portion 45 and a fitment 60 inserted within the neck 25 of the reservoir 15 which is used to retain the wick 30 such that the lower portion 40 is in fluid communication with said volatile liquid 20 and an upper portion 45 of the wick is in fluid communication with an ambient environment. According to the present embodiment, there is also present a vent 90, and as the lagging, an absorbent washer 95 interposed between the vent 90 and the ambient environment. According to this embodiment, the absorbent washer 95 functions to absorb the flow of the volatile liquid 20 in case the dispensing device is tilted or displaced from its vertical orientation (as shown by arrow “A”) to a horizontal orientation (as shown by arrow “B”) or is otherwise inverted such that the volatile liquid 20 is permitted to flow into the vent 90. Due to the presence of the absorbent washer 95 however, any volatile liquid 20 exiting the reservoir 15 and the vent 90 would become entrained in the absorbent washer 95 and would eventually evaporate or volatilize into the ambient environment. Further, as the absorbent washer 95 is not pressure-tight it would permit for the equalization of pressure between the ambient environment, and the interior of the reservoir 15 such that the relative pressure between the ambient and the interior atmosphere within the reservoir may be equalized.

FIG. 6A depicts an alternative dispensing device 120 according to the second aspect of the invention. The dispensing device 120 includes a reservoir 15 containing a volatile liquid 20, a porous wick 30 having a lower portion 40 and an upper portion 45 and a fitment 60 inserted within the neck 25 of the reservoir 15 which is used to retain the wick 30 such that the lower portion 40 is in fluid communication with said volatile liquid 20 and an upper portion 45 of the wick is in fluid communication with an ambient environment. According to the present embodiment, there is also present a vent 90 indicated by a dotted line, and as the lagging, an absorbent washer 95 interposed between the vent and the ambient environment. As described previously with reference to FIG. 5 the lagging functions to absorb the flow of volatile liquid 20 in case the dispensing device 120 is tilted or displaced from its vertical orientation (as shown by arrow “A”) to a horizontal orientation (as shown by arrow “B”) or is otherwise inverted such that the volatile liquid 20 is permitted to flow into the vent. Likewise, should any volatile liquid 20 exit the reservoir 15 via the vent, it would become entrained in the absorbent washer 95 and would eventually evaporate or volatilize into the ambient environment. Further, as the absorbent washer 95 is not pressure-tight, it would permit for pressure equalization between the ambient environment and the interior of the reservoir 15. FIG. 6B depicts a cross-section of the lower portion 40 of the wick and to the fitment 60 and to the vent 90; the vent is a recess within the fitment 60 and which runs along the length of the fitment 60. In the particular embodiment in FIG. 6B, the vent 90 is bounded on one side by the wick 40. Viewing now FIG. 6A hand 6B together, it will be understood that the vent 90 extends from within the reservoir 15 and provides means for the passage of air along the length of the fitment 60 to the lagging, here the absorbent washer 95.

FIG. 7A depicts a further alternative dispensing device 140 according to the second aspect of the invention. The dispensing device 120 includes a reservoir 15 containing a volatile liquid 20, a porous wick 30 having a lower portion 40 and an upper portion 45 and a fitment 60 inserted within the neck 25 of the reservoir 15 which is used to retain the wick 30 such that the lower portion 40 is in fluid communication with said volatile liquid 20 and an upper portion 45 of the wick is in fluid communication with an ambient environment. According to the present embodiment, there is also present at least one vent, here two vents are visible 90A and 90B and as the lagging, an absorbent washer 95 interposed between the vent and the ambient environment. As described previously with reference to FIGS. 5 and 6A the lagging functions to absorb the flow of volatile liquid 20 in case the dispensing device 140 is tilted or displaced from its vertical orientation (as shown by arrow “A”) to a horizontal orientation (as shown by arrow “B”) or is otherwise inverted such that the volatile liquid 20 is permitted to flow into the vent 90A, 90B. Likewise, should any volatile liquid 20 exit the reservoir 15 via the vent 90A, 90B it would become entrained in the absorbent washer 95 and would eventually evaporate or volatilize into the ambient environment. Further, as the absorbent washer 95 is not pressure-tight, it would permit for pressure equalization between the ambient environment and the interior of the reservoir 15. FIG. 7B depicts a cross-section of the lower portion 40 of the wick and the fitment 60 containing the two vents 90A, 90B. As illustrated the vents 90A, 90B are two passages which runs along the length of the fitment 60. Viewing now FIGS. 6A and 6B, it will be understood that the vent 90 extends from within the reservoir 15 and provides means for the passage of air along the length of the fitment 60 to the lagging, here the absorbent washer 95. It is to be understood that one or more vents may be present in either of the either of the embodiments according to the second aspect of the invention.

FIG. 8 depicts a dispensing device according to the third aspect of the invention. The dispensing device 160 shares certain elements in common with prior aspects of the invention. The dispensing device 160 includes a reservoir 15 containing a volatile liquid 20, a porous wick 30 having a lower portion 40 adapted to be in fluid communication with said volatile liquid and an upper portion 45 adapted to be in fluid communication with an ambient environment, a lagging 50 in contact with, but preferably surrounding at least a part of the porous wick 30, a sleeve 55 encasing at least a part of the said lagging 50 said sleeve including a vent 90 between the reservoir 15 and the ambient environment, and a disruptable seal 65 which in a first, sealing relationship provides a liquid tight barrier between the wick 30 and the volatile liquid 15, and in a second, non-sealing relationship permits for fluid communication between the wick 30 and the volatile liquid in the reservoir 15. Optionally the dispensing device 160 may also include further lagging, preferably in the form of an absorbent washer 95 interposed between the vent 90 and the ambient environment. This optional element is illustrated by dashed lines in FIG. 8.

FIG. 9 depicts a dispensing device according to the fourth aspect of the invention. Therein is provided a dispensing device 180 comprising which shares certain common elements with prior aspects of the invention. The dispensing device 180 includes a reservoir 15 containing a volatile liquid 20, a porous wick 30 having a lower portion 40 adapted to be in fluid communication with said volatile liquid and an upper portion 45 adapted to be in fluid communication with an ambient environment, a vent 90 between the reservoir 15 and the ambient environment. The dispensing device 180 further includes a removable overcap 190 which in a closed relationship with the reservoir 15, provides a vapor tight barrier between the upper portion 45 of the porous wick 30 and the ambient environment as well as provides a liquid tight seal between at least a portion of the overcap 190 and the vent 90. With specific reference to FIG. 9, there is provided a fitment 60 which is inserted within the neck 25 of the reservoir 15; the fitment functions to provide a support for the porous wick 30. The fitment also includes at least one vent 90 passing therethrough such that when the overcap 190 is removed, the vent 90 permits for pressure equalization between the ambient environment and the interior of the reservoir 15. The removable overcap 190 includes a contact face 192 which abuts the top surface 62 of the fitment 60. When the removable overcap 190 is in a closed relationship with the reservoir 15, its contact face 192 abuts the top surface 60 of the fitment 62 and forms an liquid tight seal with the vent 90, thereby denying the passage of any of the volatile liquid 20 out from the vent 90 should be dispensing device 180 be tilted or inverted.

FIG. 10 depicts an alternative embodiment of a dispensing device according to the fourth aspect of the invention. Therein is provided a dispensing device 180 comprising which shares certain common elements with prior aspects of the invention. The dispensing device 180 includes a reservoir 15 containing a volatile liquid 20, a porous wick 30 having a lower portion 40 adapted to be in fluid communication with said volatile liquid and an upper portion 45 adapted to be in fluid communication with an ambient environment, a vent 90 within the porous wick 30 between the reservoir 15 and the ambient environment. The dispensing device 180 further includes a removable overcap 190 which in a closed relationship with the reservoir 15, provides a vapor tight barrier between the upper portion 45 of the porous wick 30 and the ambient environment as well as provides a liquid tight seal between at least a portion of the overcap 190 and the vent 90. With specific reference to FIG. 10, there is provided a fitment 60 which is inserted within the neck 25 of the reservoir 15; the fitment functions to provide a support for the porous wick 30. The vent 90 permits for pressure equalization between the ambient environment and the interior of the reservoir 15. The removable overcap 190 includes a contact plug 194 which abuts the vent 90. When the removable overcap 190 is in a closed relationship with the reservoir 15, the contact plug 194 forms an liquid tight seal with the vent 90, thereby denying the passage of any of the volatile liquid 20 out from the vent 90 should be dispensing device 180 be tilted or inverted.

FIGS. 11A-C depict three vented wicks at can be used in the various embodiments of the present invention. All three involve a wick that fits within a sleeve of any suitable impermeable material, such as glass or plastics. In FIG. 11A, a vent is provided by means of a longitudinal groove 301 of semicircular cross-section provided in the side of a cylindrical wick 302. When this wick is inserted into a sleeve 303, the sleeve and the groove form a vent. In FIGS. 11B and 11C, the wick consists of two hemi-cylindrical portions that fit together to form a cylinder. In FIG. 11B, a longitudinal groove 304 is provided in a flat face of one hemi-cylindrical wick portion 305, and the combining of this hemi-cylindrical portion with a second hemi-cylindrical portion 306 having no groove in its flat face within a sleeve (not shown) forms a vent of seimcircular cross-section. In FIG. 11C, the flat faces of both hemi-cylindrical portions have corresponding longitudinal grooves of semicircular cross-section, such that the combination of the two portions within a sleeve (not shown) forms a vent 307 of circular cross-section. The skilled person will readily be able to ascertain suitable embodiments of the invention in which these vented wicks may be usefully used.

With regard to the various elements of the dispensing devices according to the present invention, the fitment 60, the sleeve 55 and the reservoir 15 may be produced from any material which can be suitably formed or shaped into forms appropriate to fulfilling the functions described above. Exemplary materials are non-porous materials which, by way of non-limiting example, include glass, ceramic materials, metals, as well as various polymers including high density polyolefins especially high density polyethylene, polyalkylene terephthalates, such as polyethylene terephthalate, polyvinyl chloride, Berex®, as well as other materials which are not deleteriously affected by the volatile liquid. Desirably the reservoir 15 is formed of a clear or translucent material, which may be optionally colored such as a glass or suitable polymer. The fitment 60 and sleeve 55 may be formed as a single unit, or formed from discrete parts which are thereafter assembled. Polymeric materials are particularly suited for forming the fitment 60 and sleeve 55 as such are typically readily moldable, although of course other materials may be used as well.

With regard to the reservoir, 15, the reservoir 15 may be of any shape, form or configuration which is suited to the containment of the volatile liquid which is to be provided therein. Thus, various shapes and configurations of the reservoir 15 may be based on aesthetic reasons having pleasing forms and shapes may be produced.

With regard to the wick which is included in the dispensing devices according to the present invention, the wick may be formed of known art materials. Such materials include, by way of non-limiting example, those based on natural or synthetic fibers, woven or non-woven fabrics, cords, rods or other articles, porous media such a those based on celluloses such as cardboard-based and paper-based wicks, graphite and carbonaceous wicks, as well as porous synthetic polymers and porous ceramic materials. Such wicks are commercially available from a variety of sources, including porous synthetic polymers from Porex Corp., (Fairburn, Ga., USA) and Micropore Plastics Inc. (Stone Mountain, Ga.) as well as porous ceramics available from Rauschert GmbH & Co.-KG (Germany). With regard to the wick, it is only required that it be capable of absorbing the volatile liquid 20 in the reservoir 15, and transport it by means of capillary action.

With regard to the lagging, this element may be formed of any absorbent material which will have sufficient absorbency in order to absorb a sufficient volume of the volatile liquid originally disposed in the reservoir when both the wick and lagging come into liquid contact with the volatile liquid such that the remaining volume of volatile liquid in the reservoir is insufficient to contact the lagging or the wick when the dispensing device is placed in a horizontal orientation. It is to be understood that according to especially preferred embodiments, the unit absorbency (i.e., the absorbency per unit mass and/or unit volume) of the lagging is at least equal to, or is preferably greater than the unit absorbency of the wick. Unlike the wick however, the lagging need not be a material which provides capillary transport to an absorbed liquid, but may merely be an absorbent material.

It is to be understood that in accordance with the first and third aspects of the invention, that careful consideration of the volumetric capacity of the lagging, and of the wick, as well as the relative dimensions of the wick and lagging be taken into consideration such that after disruption of the disruptable seal, they will absorb a sufficient amount of the volatile originally disposed in the reservoir such that the remaining volume of volatile liquid in the reservoir is insufficient to contact the lagging or the wick when the dispensing device is placed in a horizontal orientation.

With regard to the disruptable seal this element may be formed from any non-porous material. Non-limiting examples of suitable non-porous materials include films, foils, single- or multi-layered laminates formed from synthetic polymers, metals, metallic foils, or combinations of two or more such materials. The disruptable seal element may be conveniently affixed to, or inserted within the sleeve so to deny fluid contact between the volatile liquid, and both the lower portion of the porous wick, and with the lower end of the lagging. This disruptable seal element by affixed by any physical method (physical insertion, crimping, etc.) or by the use of a chemical agent particularly an adhesive substance interposed between at least portions of the disruptable seal element and the wall. Preferably however the disruptable seal is formed of a film which can be pierced or breached by the displacement of the wick, which wick is formed of a material having sufficient rigidity and strength so to be capable of piercing or breaching the disruptable seal.

The volatile liquid which may be used in the dispensing devices taught herein may be any volatile or evaporable material, but is desirably one or more liquids which have a cosmetic effect such as a fragrancing or odor masking effect such as may be based on one or more essential oils, or volatile liquids which have an insecticidal effect or a medical effect.

By way of non-limiting example, useful essential oils include one or more of: anethole 20/21 natural, aniseed oil china star, aniseed oil globe brand, balsam (Peru), basil oil (India), black pepper oil, black pepper oleoresin 40/20, Bois de Rose (Brazil) FOB, borneol flakes (China), camphor oil, canaga oil (Java), cardamom oil, cassia oil (China), cedarwood oil (China), cinnamon bark oil, cinnamon leaf oil, citronella oil, clove bud oil, clove leaf, coriander (Russia), coumarin (China), cyclamen aldehyde, diphenyl oxide, ethyl vanillin, eucalyptol, eucalyptus oil, eucalyptus citriodora, fennel oil, geranium oil, ginger oil, ginger oleoresin (India), white grapefruit oil, guaiacwood oil, gurjun balsam, heliotropin, isobornyl acetate, isolongifolene, juniper berry oil, L-methyl acetate, lavender oil, lemon oil, lemongrass oil, lime oil, litsea cubeba oil, longifolene, menthol, methyl cedryl ketone, methyl chavicol, methyl salicylate, musk ambrette, musk ketone, musk xylol, nutmeg oil, orange oil, patchouli oil, peppermint oil, phenyl ethyl alcohol, pimento berry oil, pimento leaf oil, rosalin, sandalwood oil, sandenol, sage oil, clary sage, sassafras oil, spearmint oil, spike lavender, tagetes, tea tree oil, vanilin, vetyver oil (Java), wintergreen.

These and other suitable materials which may be useful in the volatile liquid may be commercially obtained from a variety of suppliers including: Givaudan Corp. (Teaneck, N.J.); Berje Inc. (Bloomfield, N.J.); BBA Aroma Chemical Div. of Union Camp Corp. (Wayne, N.J.); Firmenich Inc. (Plainsboro N.J.); Quest International Fragrances Inc. (Mt. Olive Township, N.J.); Robertet Fragrances Inc. (Oakland, N.J.), or from other suppliers not necessarily listed herein.

The volatile liquids may be provided in the form of neat compositions, or may be provides as aqueous mixtures, organic mixtures or aqueous-organic mixtures which include of one or more volatile or evaporable materials.

Although not shown in several of the Figures, several further elements and features are contemplated as clearly falling within the scope of the invention. One such element is a removable overcap which may be used to encase at least the upper part of the wick such that, subsequent to assembly and prior to use, the consumer necessarily removed the overcap (which is typically discarded) thereby exposing the wick to the ambient environment, thereby initiating the delivery of volatile liquid thereto. Such an overcap may be removably affixed by any conventional means, such as by the use of corresponding mating threads on both the overcap and at least a portion of the reservoir, typically in the neck region thereof. Alternately such an overcap may be merely friction fitted to the reservoir, such that a good seal is formed between a part of the overcap and a part of the reservoir.

The dispensing devices according to the invention are activated by removal of the overcap (when present) and by permitting the volatile liquid to transfer via capillary action up the porous wick from the lower part to the upper part, from whence it is permitted to volatilize into an ambient environment. Where an embodiment includes a disruptable seal as described herein, subsequent to the removal of the overcap (when present) the wick is displaced so to disrupt the disruptable seal and thereby permit fluid communication between the wick and the volatile liquid. Thereafter, the volatile liquid to transfers via capillary action up the wick and volatilize into an ambient environment as described above.

The dispensing device according to the invention may be used to dispense a volatile liquid in and of itself, or the dispensing device may be used in conjunction with a further apparatus such as in a heated vaporizer device wherein at least a portion of the dispensing device is heated so to controllably induce the rate of delivery of the volatile liquid to the ambient environment. In conjunction with such a heated vaporizer device the dispensing device is particularly conveniently used as a refill for said heated vaporizer device. Similarly, the dispensing device may be used in conjunction with a further apparatus such as in a device which induces an airflow, such as by the use of a fan or other device, wherein at least a portion of the dispensing device, usually the wick is inserted in the induced airflow so to controllably induce the rate of delivery of the volatile liquid to the ambient environment.

The dispensing device according to the invention may be used to dispense a volatile liquid in any ambient environment. Non-limiting examples of ambient environments include interior spaces of buildings or other structures such as rooms, hallways, elevators, common areas, closets, as well as the interior spaces of vehicles such as automobile interiors, boat interiors, aircraft cabins, and the like.

While the invention is susceptible of various modifications and alternative forms, it is to be understood that specific embodiments thereof have been shown by way of example in the drawings which are not intended to limit the invention to the particular forms disclosed; on the contrary the intention is to cover all modifications, equivalents and alternatives falling within the scope and spirit of the invention as expressed in the appended claims.

1. A dispensing device for the delivery of a volatile liquid to an ambient environment comprising:
a reservoir containing a volatile liquid;
a porous wick having a lower portion adapted to be in fluid communication with said volatile liquid and an upper portion adapted to be in fluid communication with an ambient environment;
a lagging in contact at least a part of the porous wick;
a sleeve encasing at least a part of the lagging;
a disruptable seal which in a first, sealing relationship provides a liquid tight barrier between the wick and the volatile liquid, and in a second, non-sealing relationship permits for fluid communication between the wick and the volatile liquid in the reservoir.

2. A dispensing device for the delivery of a volatile liquid to an ambient environment comprising:
a reservoir containing a volatile liquid;
a porous wick having a lower portion in fluid communication with said volatile liquid and an upper portion in communication with an ambient environment;
a fitment which includes a vent between the reservoir and the ambient environment; and,
a lagging interposed between the vent and the ambient environment.

3. A dispensing device for the delivery of a volatile liquid to an ambient environment comprising:
a reservoir containing a volatile liquid;
a porous wick having a lower portion adapted to be in fluid communication with said volatile liquid and an upper portion adapted to be in fluid communication with an ambient environment;
a lagging in contact with at least a part of the porous wick;
a sleeve encasing at least a part of the said lagging, said sleeve including a vent between the reservoir and the ambient environment; and,
a disruptable seal which in a first, sealing relationship provides a liquid tight barrier between the wick and the volatile liquid, and in a second, non-sealing relationship permits for fluid communication between the wick and the volatile liquid in the reservoir.

4. A dispensing device for the delivery of a volatile liquid to an ambient environment comprising:
a reservoir containing a volatile liquid;
a porous wick having a lower portion adapted to be in fluid communication with said volatile liquid and an upper portion adapted to be in fluid communication with an ambient environment;
a vent between the reservoir and the ambient environment; and,
an overcap which in a closed relationship provides a vapor tight barrier between the upper portion of the porous wick and the ambient environment as well as provides a liquid tight seal between at least a portion of the overcap and the vent.
